(19) United States
(12) Patent Application Publication
(10) Pub. No.: US 2026/0053427 A1
(43) Pub. Date: Feb. 26, 2026

(54) ULTRASOUND METHODS AND DEVICES FOR ENHANCING CYTOPATHOLOGIC CELL COLLECTION

(71) Applicant: Adenocyte Ltd., Jerusalem (IL)

(72) Inventors: Mark RUTENBERG, Jerusalem (IL); Dan ADAM, Haifa (IL); Kyle MORRISON, Bothell, WA (US)

(73) Assignee: Adenocyte Ltd., Jerusalem (IL)

(21) Appl. No.: 19/375,375

(22) Filed: Oct. 31, 2025

Related U.S. Application Data
(63) Continuation of application No. PCT/IL2024/050424, filed on May 2, 2024.
(60) Provisional application No. 63/463,344, filed on May 2, 2023.

(51) Int. Cl. A61B 5/00; A61B 10/02; A61B 17/22

(52) U.S. Cl. CPC A61B 5/4381; A61B 10/02; A61B 17/22004; A61B 2017/22005

(57) ABSTRACT

A method of producing cytopathologic cell samples, the method including insonating a target tissue by ultrasound energy with a focused beam at that generates shear waves within the tissue. A method of producing cytopathologic cell samples, the method including insonating a target tissue by ultrasound energy at a first intensity level during a first period, insonating the target tissue by ultrasound energy at a second intensity level during a second period, wherein the first intensity level is higher than the second intensity level. Related apparatus and methods are also described.

### RELATED APPLICATIONS

This application is a Continuation of PCT Patent Application No. PCT/IL2024/050424 having International filing date of May 2, 2024 which claims the benefit of priority 35 USC § 119 (e) of U.S. Provisional Patent Application No. 63/463,344 filed on May 2, 2023. The contents of the above applications are all incorporated by reference as if fully set forth herein in their entirety.

### FIELD OF THE INVENTION

The present disclosure, in some embodiments thereof, relates to medical systems and methods for separating targeted epithelium and in particular to an ultrasound method for separating targeted epithelium, and to ultrasound devices constructed and/or programmed to use such methods, and to programming ultrasound devices to use such methods.

### BACKGROUND OF THE INVENTION

Pancreatic cancer is an extremely deadly cancer whose high mortality rate stems primarily from its tendency to be detected at an advanced stage. The absence of reliable and/or non-invasive tests for early detection of pancreatic cancer is responsible for its late detection. There have been attempts to harvest pancreatic cells in pancreatic juice for purposes of early detection, but such tests suffer from low sensitivity due to the paucity of pancreatic cells that are naturally found in pancreatic juice.

Additional background art includes:

U.S. patent application Ser. No. 17/007,953, filed on Aug. 31, 2020, titled Device for Inducing Exfoliation of Cells and/or Tissue Fragments for Enhanced Cytopathologic Cell Collection.

The disclosures of all references mentioned above and throughout the present specification, as well as the disclosures of all references mentioned in those references, are hereby incorporated herein by reference.

### SUMMARY

The present disclosure, in some embodiments thereof, relates to an ultrasound method for breaking up targeted epithelium by using ultrasound shear waves, to ultrasound devices constructed and/or programmed to use such methods, and to programming ultrasound devices to use such methods.

The present disclosure, in some embodiments thereof, relates to combining ultrasound methods for enhancing exfoliation of cells and/or molecules of a targeted epithelium, to ultrasound devices constructed and/or programmed to use such methods, and to programming ultrasound devices to use such methods.

A broad aspect of some embodiments of the invention includes insonating tissue with high intensities of focused ultrasound waves during a period of time to generate shear waves that partially segment the target epithelium by creating lateral breaks between cells in order to induce exfoliation of cells and tissue fragments.

A broad aspect of some embodiments of the invention includes insonating tissue with higher intensities of focused ultrasound waves during a first period of time to generate shear waves that partially segment the target epithelium by creating lateral breaks between cells, followed by injection of a microbubble contrast agent and insonating the tissue with lower intensities of ultrasound waves during a second period of time that lifts the partially segmented epithelium from below, in order to induce exfoliation of cells and tissue fragments.

According to an aspect of some embodiments of the present disclosure there is provided a method of inducing exfoliation of cells and molecules from a targeted epithelium, the method including insonating a target tissue with a high energy mechanical index (MI) at a level greater than 0.4 focused beam to generate shear waves within the tissue.

According to an aspect of some embodiments of the present disclosure there is provided a method of producing cytopathologic cell samples, the method including insonating a target tissue by ultrasound energy with a focused beam that generates shear waves within the tissue.

According to an aspect of some embodiments of the present disclosure there is provided a method of inducing exfoliation of cells and molecules from a targeted epithelium, the method including application of a focused ultrasound beam, wherein the ultrasound beam is arranged to produce a mechanical index (MI) in a range between 0.4 and 1.8, to produce a beam width at focus in a range between 2 millimeters and 10 millimeters, to be produced at a pulse width in a range between 100 microseconds and 800 microseconds, and to be produced at a pulse repetition rate in a range between 4 Hz and 20 Hz.

According to an aspect of some embodiments of the present disclosure there is provided a method of producing cytopathologic cell samples, the method including insonating a target tissue by ultrasound energy at a first intensity level during a first period, insonating the target tissue by ultrasound energy at a second intensity level during a second period, wherein the first intensity level is higher than the second intensity level.

According to some embodiments of the disclosure, the first intensity level is at a Mechanical Index (MI) in a range between 0.3 and 1.8.

According to some embodiments of the disclosure, insonating of the target tissue by ultrasound energy during the first period includes insonating by a focused ultrasound beam.

According to some embodiments of the disclosure, the second intensity level is at a Mechanical Index (MI) in a range between 0.1 and 0.4.

According to some embodiments of the disclosure, the insonating of the target tissue by ultrasound energy during the second period includes insonating by a non-focused ultrasound beam.

According to some embodiments of the disclosure, the insonating of the target tissue by ultrasound energy during the FIRST period includes insonating by repeating ultrasound pulses of a pulse width between 2 μS and 800 μS.

According to some embodiments of the disclosure, the insonating of the target tissue by ultrasound energy during the SECOND period includes insonating by repeating ultrasound pulses of a duration between 20 μS and 800 μS.

According to some embodiments of the disclosure, the insonating the target tissue by ultrasound energy during the first period includes repeating the ultrasound pulses at a rate in a range between 1 to 20 times per second.

According to some embodiments of the disclosure, the insonating the target tissue by ultrasound energy during the second period includes repeating the ultrasound pulses at a rate in a range between 1 to 100 times per second.

According to some embodiments of the disclosure, the insonating the target tissue by ultrasound energy at the second intensity level during the second period includes providing ultrasound contrast agent to the target tissue before the insonating the target tissue by ultrasound energy at the second intensity level during the second period.

According to some embodiments of the disclosure, the insonating the target tissue by ultrasound energy during the second period includes providing ultrasound contrast agent to the target tissue during the insonating the target tissue by ultrasound energy during the second period.

According to some embodiments of the disclosure, the target tissue includes a pancreas.

According to some embodiments of the disclosure, the target tissue includes a prostate. According to other embodiments of the disclosure, the target tissue includes a bladder.

According to some embodiments of the disclosure, further including injecting a patient with a drug which induces pancreatic secretion, and collecting the cytopathologic samples by collecting pancreatic secretion.

According to some embodiments of the disclosure, the target tissue includes a body location selected from a group consisting of a mediastinum, a pleura, a pericardium, a peritoneum, a lung, a breast, salivary glands, a meninges, a pancreas, a pancreatic duct, a pancreatic cyst, a kidney, a liver, a prostate, a bladder, and ovaries.

According to an aspect of some embodiments of the present disclosure there is provided a method of inducing exfoliation, the method including insonating a target tissue by ultrasound energy at a Mechanical Index (MI) in a range between 1.2 and 1.8.

According to some embodiments of the disclosure, the insonating the target tissue by ultrasound energy includes insonating by a focused ultrasound beam.

According to some embodiments of the disclosure, the insonating the target tissue by ultrasound energy includes insonating by repeating ultrasound pulses of a pulse width between 20 μS and 800 μS.

According to some embodiments of the disclosure, the insonating the target tissue by ultrasound energy includes repeating the ultrasound pulses at a rate in a range between 1 to 50 times per second.

According to an aspect of some embodiments of the present disclosure there is provided an ultrasound energy source programmed to insonate using an insonation procedure, the procedure including insonating using focused ultrasound energy at a first intensity level designed to produce shear waves in insonated tissue during a first period, followed by insonating using unfocused ultrasound energy at a second intensity level during a second period, wherein the second intensity level is lower than the first intensity level.

According to an aspect of some embodiments of the present disclosure there is provided a program for controlling an ultrasound source, the program including insonating using ultrasound energy at a first intensity level during a first period, followed by insonating using ultrasound energy at a second intensity level during a second period, wherein the first intensity level is higher than the second intensity level.

According to an aspect of some embodiments of the present disclosure there is provided a method of programming an ultrasound source, the method including programming an ultrasound source to insonate using ultrasound energy at a first intensity level during a first period, programming the ultrasound source to insonate using ultrasound energy at a second intensity level during a second period, wherein the first intensity level is higher than the second intensity level. Following is a non-exclusive list including some examples of embodiments of the invention. The invention also includes embodiments which include fewer than all the features in an example and embodiments using features from multiple examples, also if not expressly listed below.

Example 1. A method of inducing exfoliation of cells and molecules from a targeted epithelium, the method comprising:
insonating a target tissue with a high energy mechanical index (MI) at a level greater than 0.4 focused beam to generate shear waves within the tissue.

Example 2. The method according to example 1, wherein the insonating comprises insonating a target tissue by ultrasound energy with a focused beam at that generates shear waves within the tissue.

Example 3. The method according to examples 1 or 2 wherein the insonating comprises using an application of a focused ultrasound beam, wherein the ultrasound beam is arranged:
to produce a mechanical index (MI) in a range between 0.4 and 1.8;
to produce a beam width at focus in a range between 2 millimeters and 10 millimeters;
to be produced at a pulse width in a range between 100 microseconds and 800 microseconds; and
to be produced at a pulse repetition rate in a range between 4 Hz and 20 Hz.

Example 4. The method according to any of examples 1-3, wherein the insonating comprises:
insonating a target tissue by ultrasound energy at a first intensity level during a first period, and wherein the method further comprises insonating the target tissue by ultrasound energy at a second intensity level during a second period, wherein the first intensity level is higher than the second intensity level.

Example 5. The method according to example 4, wherein the first intensity level is at a Mechanical Index (MI) in a range between 0.3 and 1.8.

Example 6. The method according to any one of examples 4-5, wherein the insonating the target tissue by ultrasound energy during the first period comprises insonating by a focused ultrasound beam.

Example 7. The method according to any one of examples 4-6, wherein the second intensity level is at a Mechanical Index (MI) in a range between 0.1 and 0.4.

Example 8. The method according to any one of examples 4-7, wherein the insonating the target tissue by ultrasound energy during the second period comprises insonating by a non-focused ultrasound beam.

Example 9. The method according to any one of examples 4-8, wherein the insonating the target tissue by ultrasound energy during the first period comprises insonating by repeating ultrasound pulses of a pulse width between 2 μS and 800 μS.

Example 10. The method according to any one of examples 4-9, wherein the insonating the target tissue by ultrasound energy during the second period comprises insonating by repeating ultrasound pulses of a duration between 20 μS and 800 μS.

Example 11. The method according to any one of examples 9-10, wherein the insonating the target tissue by ultrasound energy during the first period comprises repeating the ultrasound pulses at a rate in a range between 1 to 20 times per second.

Example 12. The method according to any one of examples 9-11, wherein the insonating the target tissue by ultrasound energy during the second period comprises repeating the ultrasound pulses at a rate in a range between 1 to 100 times per second.

Example 13. The method according to any of examples 1-11, wherein the duration of the first period ranges between 1 to 15 minutes.

Example 14. The method according to any of examples 1-13, wherein the duration of the second period ranges between 3 to 30 minutes.

Example 15. The method according to any one of examples 4-12, wherein the insonating the target tissue by ultrasound energy at the second intensity level during the second period comprises providing ultrasound contrast agent to the target tissue before the insonating the target tissue by ultrasound energy at the second intensity level during the second period.

Example 16. The method according to any one of examples 4-15, wherein the insonating the target tissue by ultrasound energy during the second period comprises providing ultrasound contrast agent to the target tissue during the insonating the target tissue by ultrasound energy during the second period.

Example 17. The method according to any of examples 4-16, wherein the duration of the second period ranges between 10 to 20 minutes.

Example 18. The method according to any one of examples 4-16, wherein the target tissue comprises a pancreas.

Example 19. The method according to example 18, and further comprising injecting a patient with a drug which induces pancreatic secretion, and collecting the cytopathologic samples by collecting pancreatic secretion.

Example 20. The method according to any one of examples 4-18, wherein the target tissue comprises a body location selected from a group consisting of:
a mediastinum;
a pleura;
a pericardium;
a peritoneum;
a lung;
a breast;
salivary glands;
a meninges;
a pancreas;
a pancreatic duct;
a pancreatic cyst;
a prostate;
a kidney;
a liver;
a bladder; and
ovaries.

Example 21. The method according to example 20, wherein the target tissue comprises a prostate, and wherein the method comprises collecting one or more of urine and ejaculate.

Example 22. The method according to example 20 or 21, comprising promoting urination by the patient by one or more of administrating the patient with a diuretic substance and prompting the patient to consume fluids.

Example 23. A method of inducing exfoliation, the method comprising insonating a target tissue by ultrasound energy at a Mechanical Index (MI) in a range between 1.2 and 1.8.

Example 24. The method according to example 23, wherein the insonating the target tissue by ultrasound energy comprises insonating by a focused ultrasound beam.

Example 25. The method according to any one of examples 23-24, wherein the insonating the target tissue by ultrasound energy comprises insonating by repeating ultrasound pulses of a pulse width between 20 μS and 800 μS.

Example 26. The method according to example 25, wherein the insonating the target tissue by ultrasound energy comprises repeating the ultrasound pulses at a rate in a range between 1 to 50 times per second.

Example 27. An ultrasound energy source programmed to insonate using an insonation procedure, the procedure comprising:
selecting program parameters to produce shear waves for causing or assisting cells exfoliation; and
insonating using focused ultrasound energy at a first intensity level designed to produce shear waves in insonated tissue during a first period.

Example 28. The ultrasound energy source according to example 27, wherein said insonating is followed or preceded by
insonating using unfocused ultrasound energy at a second intensity level during a second period, wherein the second intensity level is lower than the first intensity level.

Example 29. A program for controlling an ultrasound source, the program comprising:
insonating using ultrasound energy at a first intensity level during a first period; followed by
insonating using ultrasound energy at a second intensity level during a second period;

wherein
the first intensity level is higher than the second intensity level.

Example 30. A method of programming an ultrasound source, the method comprising:
programming an ultrasound source to insonate using ultrasound energy at a first intensity level during a first period;
programming the ultrasound source to insonate using ultrasound energy at a second intensity level during a second period;
wherein
the first intensity level is higher than the second intensity level.

Example 31. An isolated sample of a body fluid obtained from an insonated prostate of a subject, wherein the isolated sample comprises exfoliated cells in sufficient quantity for analysis and detection of abnormal cells.

Example 32. The isolated sample of a body fluid according to example 31, comprising one or more of urine and ejaculate.

Example 33. The isolated sample of a body fluid according to example 31 or 32, comprising contiguous cells from the prostate.

Example 34. The isolated sample of a body fluid according to example 33, wherein the contiguous cells comprise a fragment of epithelia tissue or a sheet of cells.

Example 35. The isolated sample of a body fluid according to example 34, comprising more than one epithelia tissue fragment.

Example 36. The isolated sample of a body fluid according to example 34 or 35, comprising intact epithelia tissue fragments from the organ.

Example 37. The isolated sample of a body fluid according to any one of the preceding examples, wherein the isolated sample is obtained by insonating the prostate with high intensities of focused ultrasound waves.

Example 38. An ultrasound device programmed to insonate a prostate, comprising at least one probe for insonating the prostate, wherein the at least one probe comprises one or more of a transrectal ultrasound probe, a transperineal ultrasound probe and a ultrasound catheter probe.

Example 39. The ultrasound device programmed to insonate a prostate according to example 38, comprising an electroejaculation device.

Example 40. The ultrasound device programmed to insonate a prostate according to example 38 or 39, comprising a prostate massager.

Example 41. The ultrasound device programmed to insonate a prostate according to any of examples 38-39, programmed to:
selecting program parameters to produce shear waves for causing or assisting cells exfoliation; and
insonating using focused ultrasound energy at a first intensity level designed to produce shear waves in insonated tissue during a first period.

Example 42. The ultrasound device programmed to insonate a prostate according to example 41, wherein said insonating is followed or preceded by
insonating using unfocused ultrasound energy at a second intensity level during a second period, wherein the second intensity level is lower than the first intensity level.

Example 43. A method of inducing exfoliation, the method comprising insonating a target tissue by ultrasound energy at a Mechanical Index (MI) in a range between 1.2 and 1.8.

Example 44. The method according to example 43, wherein the insonating the target tissue by ultrasound energy comprises insonating by a focused ultrasound beam.

Example 45. The method according to any one of examples 43-44, wherein the insonating the target tissue by ultrasound energy comprises insonating by repeating ultrasound pulses of a pulse width between 20 μS and 800 μS.

Example 46. The method according to example 45, wherein the insonating the target tissue by ultrasound energy comprises repeating the ultrasound pulses at a rate in a range between 1 to 50 times per second.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present disclosure may be embodied as a system, method or computer program product. Accordingly, some embodiments of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present disclosure may take the form of a computer program product embodied in one or more computer readable medium(s) having computer readable program code embodied thereon. Implementation of the method and/or system of some embodiments of the disclosure can involve performing and/or completing selected tasks manually, automatically, or a combination thereof. Moreover, according to actual instrumentation and equipment of some embodiments of the method and/or system of the disclosure, several selected tasks could be implemented by hardware, by software or by firmware and/or by a combination thereof, e.g., using an operating system.

For example, hardware for performing selected tasks according to some embodiments of the disclosure could be implemented as a chip or a circuit. As software, selected tasks according to some embodiments of the disclosure could be implemented as a plurality of software instructions being executed by a computer using any suitable operating system. In an exemplary embodiment of the disclosure, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as performing a combination of ultrasound methods for enhancing cytopathologic cell collection, might be expected to use completely different methods, e.g., making use of expert knowledge and/or the pattern recognition capabilities of the human brain, which may be more efficient than manually going through the steps of the methods described herein.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the disclosure are herein described, by way of example only, with reference to the accompanying drawings and images. With specific reference now to the drawings and images in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIG. 1A is a simplified qualitative illustration of insonation which is used to cause exfoliation of cytopathologic cell samples according to an example embodiment of the invention;

FIG. 1B is a simplified qualitative illustration of insonation during a procedure for producing cytopathologic cell samples according to an example embodiment of the invention;

FIG. 1C is a simplified qualitative illustration of levels of insonation during a procedure for producing cytopathologic cell samples according to an example embodiment of the invention;

FIG. 1D is a simplified qualitative illustration of levels of insonation during a procedure for producing cytopathologic cell samples according to an example embodiment of the invention;

FIG. 2A is a simplified flow chart illustration of a method for producing cytopathologic cell samples according to an example embodiment of the invention;

FIG. 2B is a simplified flow chart illustration of a method for producing cytopathologic cell samples according to an example embodiment of the invention;

FIG. 2C is a simplified flow chart illustration of a method for producing cytopathologic cell samples according to an example embodiment of the invention;

FIG. 2D is a simplified flow chart illustration of a method for inducing exfoliation according to an example embodiment of the invention;

FIG. 3A-3D are simplified illustrations of using ultrasound and ultrasound contrast agent to produce a cell sample according to an example embodiment of the invention;

FIG. 3E is a simplified illustration of using ultrasound and ultrasound contrast agent to produce a cell sample according to an example embodiment of the invention;

FIG. 4A is a front perspective view of an ultrasound probe according to an embodiment of the invention;

FIG. 4B is a schematic view of a probe configuration according to an example embodiment of the invention;

FIG. 4C is a simplified drawing of an array of elements in an ultrasound transducer according to an example embodiment of the invention;

FIG. 4D is a simplified drawing of an array of elements in an ultrasound transducer according to an example embodiment of the invention;

FIG. 5 is an image of a group of contiguous cells obtained by an example embodiment of the invention;

FIG. 6 is a flow chart of a method for producing cytopathologic cell samples from a prostate, according to an example embodiment of the invention;

FIG. 7 is a schematic cross-sectional view of a prostate follicle (or microscopic gland) and a segment of a prostate duct, according to an example embodiment of the invention;

FIG. 8 is a schematic cross-sectional view of a patient's pelvic region and simplified drawings of ultrasound probes interacting with the patient's prostate, according to various example embodiments of the invention; and

FIG. 9 is a table of experimental results embodiment of the present invention on human patients.

### DESCRIPTION OF SPECIFIC EMBODIMENTS

The present disclosure, in some embodiments thereof, relates to an ultrasound method for breaking up targeted epithelium by using ultrasound shear waves, to ultrasound devices constructed and/or programmed to use such methods, and to programming ultrasound devices to use such methods.

The present disclosure, in some embodiments thereof, relates to combining ultrasound methods for enhancing exfoliation of cells and/or molecules of a targeted epithelium, to ultrasound devices constructed and/or programmed to use such methods, and to programming ultrasound devices to use such methods.

A broad aspect of some embodiments of the invention includes insonating tissue during a period of time to potentially induce shear waves in the tissue to promote exfoliation of cells from the tissue, optionally, by promoting separation of cells and/or tissue fragments from the tissue and/or from each other. It is noted that in some embodiments, exfoliation is preferably induced without causing thermal damage and/or lasting damage to the tissue/organ. It should also be noted that, generally, cells and tissue regenerate after the exfoliation. In some embodiments, the exfoliation produces a sample comprising unruptured cells and/or intact tissue fragment(s) in a sufficient amount for detecting abnormalities.

In some embodiments, shear waves are induced by applying ultrasound waves, optionally, high intensities of focused ultrasound waves. Without being bound to theory, potentially segment the target epithelium by creating lateral breaks (e.g., disconnect) between cells. Without being bound to theory, generating shear waves in a tissue/organ potentially tends to separate abnormal (e.g., cancerous cells and/or tissue fragment(s)) and/or from each other from the organ, having the potential advantage of enhancing the reliability of the sample.

A broad aspect of some embodiments of the invention includes insonating tissue with higher intensities of focused ultrasound waves during a first period of time to generate shear waves that partially segment the target epithelium by creating lateral breaks between cells and/or their backing, followed by injection of a microbubble contrast agent and insonating the tissue with lower intensities of ultrasound waves during a second period of time that without being bound to theory separates the partially segmented epithelium from the organ surface, in order to induce exfoliation of cells and tissue fragments.

A broad aspect of some embodiments of the invention includes insonating tissue with lower intensities of ultrasound waves during a first period of time, followed by insonating the tissue with higher intensities of ultrasound waves during a second period of time, in order to induce exfoliation of cells from the tissue. In some embodiments, the higher intensities of ultrasound waves during the second period are focused ultrasound waves. In some embodiments, a microbubble contrast agent is injected prior to insonating tissue with lower intensities of ultrasound waves during the first period of time. Without being bound to theory, in some embodiments, insonating microbubbles with lower intensities of ultrasound waves promote separation of tissue fragments from the organ while higher intensities of ultrasound waves promote separation of cells from the organ. This order has the potential advantage of increasing the amount and/or size of exfoliated cells cluster(s) and/or tissue fragment(s) (e.g., sheet(s))

### Overview

Some methods and systems described herein are used for obtaining cells and tissue fragments exfoliated from target tissue, by way of a non-limiting example epithelium, potentially along with biological molecules that are associated with dysplasia and/or cancer that may also be shed from the target tissue, in greater quantity than previously observed by different means.

Some methods and systems described herein are used for obtaining contiguous cells, and/or cell groups, cell sheets, and/or tissue fragment samples, for evaluating the evaluate the structure of the epithelium from a target tissue at a greater rate, that is, number of samples per time unit than previously observed by different means.

Some methods and systems described herein are used for obtaining cell sheets, and/or cell groups and/or tissue fragment samples containing a larger number of cells in the sheet/group/fragment than previously observed by different means.

For example, a target tissue such as a pancreas is optionally insonated by ultrasound energy at a higher intensity during a first period, followed by ultrasound energy at a lower energy during a second period. There may or may not be a rest period between the first period of insonation and the second period of insonation.

For example, a target tissue such as a pancreas is optionally insonated by ultrasound energy at a lower intensity during a first period, followed by ultrasound energy at a higher energy during a second period. There may or may not be a rest period between the first period of insonation and the second period of insonation.

Shear waves, caused by high intensity, long duration (e.g., 100 μs-800 μs) pulses of focused ultrasound beams. Typically, such ultrasound beams are perpendicular to the surface of epithelium. The shear-wave-inducing ultrasound beams typically traverse through an organ and produce pressure changes that cause fast expansions and contractions that are typically parallel to a face plane of the tissue. These mechanical changes potentially produce separation among lateral connections between epithelial cells. It is noted that standard imaging ultrasound uses a pulse width of 2 μs.

An ultrasound contrast agent, which may be 1μ-4μ sized gas microbubbles, optionally encapsulated by protein, lipid or a polymeric shell, when exposed to ultrasound waves, expand and contract due to the changes in their local pressure changes. Thus, when injected intravenously to a subject, the microbubbles flow through the vascular system and all its capillaries, reaching also the rich microvasculature surrounding the Acinar cells and ductal system. Co-employment of an ultrasound beam aimed at the pancreas, and the existence of microbubbles within its microvasculature, potentially causes synchronized expansion-contraction of the microvasculature that underlies the epithelial layer thus dislocating cells and clusters of cells by disruption of cell junctions between the base of an epithelial cell and basement membrane below it.

In some examples, the first period of insonation includes insonation by focused ultrasound energy, which potentially generates shock waves in the tissue, and/or potentially induces shear waves in the tissue. In some examples, the first period, of higher energy ultrasound energy, is at a Mechanical Index (MI) in a range between 1.0 to 1.8.

In some examples, the second period of insonation includes insonation by non-focused ultrasound energy. In some examples, the second period, of lower energy ultrasound energy, is at a MI in a range between 0.1 to 0.4, or even 1.0. By way of a non-limiting examples the MI may be 0.25 or 0.3.

In some examples, an ultrasound contrast material, such as, by way of a non-limiting example, Lumison microbubbles, is provided at the target tissue, before and/or during the second period of insonation. The ultrasound contrast material interacts with the ultrasound energy, and may serve to dislodge cells and/or cell sheet samples, and may serve to carry the samples along the target tissue lumen(s) to be collected.

Various non-limiting examples of ultrasound contrast material include:
the above-mentioned Lumison;
Optison, a Food and Drug Administration (FDA)-approved microbubble, provided by GE Healthcare, which has an albumin shell and octafluoropropane gas core;
Definity, which is an FDA-approved microbubble provided by Lantheus. Definity includes perflutren lipid microspheres composed of octafluoropropane encapsulated in an outer lipid shell; and
Lumason, also known as SonoVue, provided by Bracco, which is also FDA-approved. It is a sulfur hexafluoride containing lipid-encapsulated microbubble.

In some examples, the first period and/or the second period of insonation includes insonation using ultrasound pulses of a width or pulse duration of 200 μs. In some examples, the first period and/or the second period of insonation includes insonation using ultrasound pulses of a width or pulse duration in a range between 20 μs and 800 μs, by way of a non-limiting example a pulse width of 200 μs.

In some examples, the first period and/or the second period of insonation includes insonation by repeating ultrasound pulses at a relatively high repetition of 8 times per second. In some examples, the first period and/or the second period of insonation includes insonation by repeating ultrasound pulses at a relatively high repetition rate in a range between 1 to 100 times per second, by way of a non-limiting example a repetition rate of 20 times per second (20 Hz).

It is noted that current practice, when ultrasound shear waves are used for various uses, the ultrasound shear wave are produced at a repletion rate of 1 per second, or 1 Hz. Increasing the repletion rate enables conveying more energy to the insonated tissue, potentially inducing separation of intact tissue fragments from a target organ.

In some examples the first period of insonation lasts for 5 minutes of shear wave at higher MI followed and the second period of insonation lasts for approximately 10-20 minutes of microbubbles at lower MI.

In some examples, the first period of insonation lasts in a range between 1 and 15 minutes. In some examples, the second period of insonation lasts in a range between 10 and 20 minutes. In some examples, the second period of insonation lasts in a range between 3 and 30 minutes.

Collecting cell samples is optionally done following ultrasound insonation by extraction of a body fluid that is in contact with the subject tissue that was insonated.

In some embodiments, the two periods are optionally provided in a reverse order relative to that described above. In some embodiments, the first period of insonation uses non-focused lower intensity ultrasound waves, and the second period of insonation uses higher intensity focused ultrasound energy, which potentially generates shock waves in the tissue, and/or potentially induce shear waves in the tissue. Optionally, values of pulse width, repetition rate, and MI used in the first period of insonation which uses non-focused lower intensity ultrasound waves are similar to the values described for the non-focused lower intensity ultrasound waves second period of insonation described above. Optionally, values of pulse width, repetition rate, and MI used in the second period of insonation which uses focused higher intensity ultrasound waves, which potentially generates shock waves in the tissue, and/or potentially induce shear waves in the tissue, are similar to the values described for the focused higher intensity ultrasound waves of the first period of insonation described above.

In some embodiments, secretin is used to induce pancreatic fluid secretion. Secretin takes 10-15 minutes to be maximally effective. Both secretin and an ultrasound contrast agent are administered intravenously, and typically require a caretaker to do so. Shear wave insonation typically does not require a caretaker and/or intravenous administration. In other target organs, secretin can be replaced by another drug that encourages flushing, depending on the nature of the organ. For example, saliva-enhancing materials for salivary glands.

In some embodiments, the procedures which require a caretaker, such as administering ultrasound contrast agent and performing the lower intensity non-focused insonation, and the administering secreting, are optionally performed at the same time. In some embodiments the administering and non-focused lower intensity insonation are grouped together and performed first, before the focused, higher intensity insonation.

A non-focused lower intensity insonation duration as described herein may typically last for approximately 10-15 minutes, similar to the duration required for secreting to be maximally effective, which potentially provides a useful benefit of performing both simultaneously.

### Sonoporation

An aspect of some embodiments relates to using ultrasound insonation as described herein disrupt a tissue, potentially enabling enhanced drug delivery.

In some embodiments, ultrasound insonation at the lower intensities as described herein plus microbubbles produced by ultrasound contrast agent are used to disrupt the tissue, potentially enabling enhanced drug delivery.

In some embodiments, ultrasound insonation at the higher intensities as described herein is used to disrupt the tissue, potentially enabling enhanced drug delivery.

In some embodiments, ultrasound insonation at the higher intensities as described herein, followed by ultrasound insonation at the lower intensities as described herein plus microbubbles produced by ultrasound contrast agent are used to disrupt the tissue, potentially enabling enhanced drug delivery.

In some embodiments, the reverse-order of the insonation periods is optionally used to disrupt the tissue, potentially enabling enhanced drug delivery.

### Target Organs

A feature of target organs which lend themselves to use of ultrasound methods for enhancing cytopathologic cell collection as described herein includes an organ in which the fluid in contact with the subject epithelium that was insonated has a natural anatomic exit that is accessible for its collection. By way of a non-limiting example—a glandular organ which has an exit. Some non-limiting examples include the pancreatic juice that exits the pancreas at the ampula, breast ductal fluid that exits the breast at the nipple, urine that exits the kidneys and bladder at the urethra, and prostatic fluid that exits at the urethra.

Some non-limiting examples of target organs for use of ultrasound methods for enhancing cytopathologic cell collection as described herein include:
a pancreas;
a pancreatic duct;
a pancreatic cyst;
a breast;
a prostate;
a mediastinum;
a pleura;
pericardium;
peritoneum;
a lung;
salivary glands;
meninges;
a kidney;
a liver;
a bladder; and
an ovary.

It is noted that many example embodiments provided herein are written using the pancreas as an example, however the examples are not meant to be limited to the pancreas and extend at least to the other target organs listed herein, as well as to additional organs sharing the properties listed in the first paragraph of the “Target organs” section.

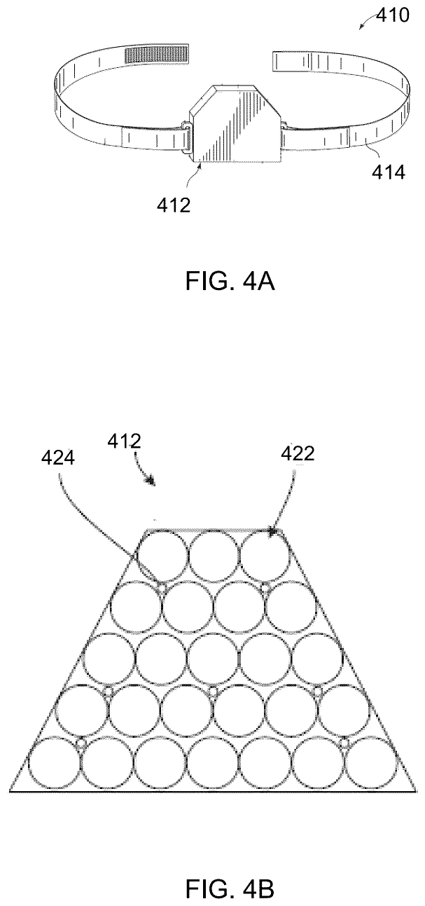

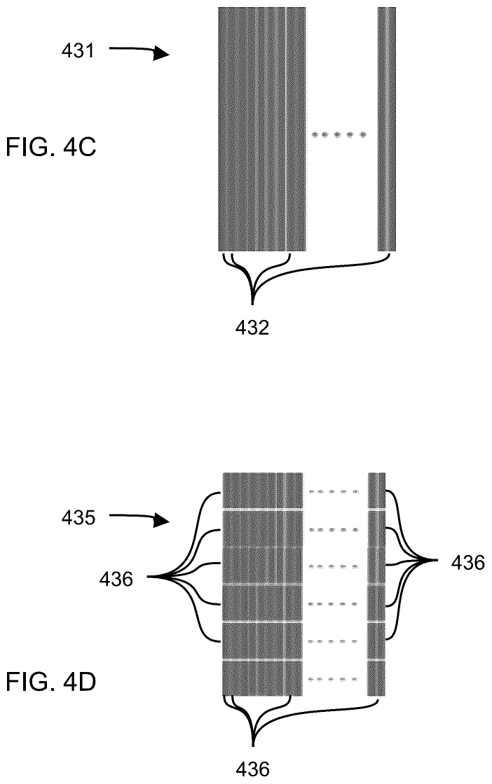

For example, In some embodiments, the target organ is a bladder. In some embodiments, the bladder should be full (e.g., with urine) and/or substantially full during ultrasound treatment, for potentially expediting sample collection with less and/or no external intervention. Alternatively or additionally, the patient is prompted to consume drinks and/or administrated with diuretic(s) to cause urination. In some embodiments, the bladder is insonated externally, optionally, using devices as described herein for the prostate, mutatis mutandis. For example, applying an ultrasound probe on the patient's skin at the lower abdomen region and/or perineum. In some embodiments, a bladder-sized patch can be used, optionally a variant of probe 410 (shown for example in FIG. 4A) with a larger contact surface of the probe. In some embodiments, the device comprises a strap (e.g., strap 414) having a size and/or shape adjusted to insonat the bladder. For example, a probe intended for the perineum is connected to a strap sized and/or sized to fit in the pelvic region.

In some embodiments, the bladder is insonated to generate shear waves in the tissue (as described in this document), having the potential advantage of reducing microbubbles amount and/or time which potentially reduces and/or prevents interaction of the microbubbles with others (e.g., surrounding) organs. Additionally, generating shear waves at the bladder potentially allows to focus the ultrasound waves at the bladder, having the potential advantage of reducing and/or avoiding damaging surrounding organs blood vessels and/or nerves by the ultrasound waves.

### Motivation

Ultrasound application according to the examples described herein may be described as insonating with tissue in two phases: 1) generating shear waves at a target tissue, optionally, focused shear waves. In some embodiments, the shear waves in the tissue are created by applying focused ultrasound waves at high MI, without microbubbles, for example, MI in a range of 1.2-1.8, or 1-1.9, or 0.5-1.7, or about MI of 1.4, or 1, or 2 or 1.6, or lower or higher or intermediate values of MI. Currently, the FDA approves MI of no more than 1.9, in some embodiments, since the ultrasound waves are not applied for imaging, higher MI values (e.g., than 1.9) may be selected.

Without being bound to theory, this induces vibration of epithelium in a plane that is roughly parallel to basement membrane, breaking lateral bonds between epithelial cells between cells, followed by: 2) a lower intensity non-focused ultrasound insonation at an MI of equal to or less than 0.4, that potentially causes intravenously introduced microbubbles in the underlying basement membrane to resonate, with a result that tissue clusters that had their lateral bonds broken during phase 1 now have their basal bonds broken, resulting in cells and large tissue clusters being lifted off from below. In some embodiments, contrast agent microbubbles are caused to oscillate within vasculature supplying the epithelial layers.

It is believed that procedures as described herein can potentially substantially increase a total number of cells to be expressed, for example pancreatic cells in pancreatic juice. Moreover, the procedures set forth herein may induce the separation of intact tissue fragments from a target organ such as the pancreas.

Such procedures, when applied to the other organs or body sites, such as, by way of some non-limiting examples, the mediastinum, pleura, pericardium, peritoneum, lung, breast, salivary glands, meninges, pancreatic ducts, pancreatic cysts, kidney, liver, bladder, or ovaries, may dramatically increase the total number of cells to be expressed in the surrounding fluids of such organs.

In some examples, the procedures set forth herein potentially induce separation of intact tissue fragments from the above-cited exemplary organs. For example, the procedures set forth herein are optionally utilized to induce exfoliation of lung cells into surrounding sputum or to induce exfoliation of bladder cells and bladder tissue into surrounding urine.

An aspect of some embodiments of the invention relates to non-invasive early detection of pancreatic cancer and/or dysplasia. Pancreatic cancer is an extremely deadly cancer primarily because it is frequently diagnosed at an advanced stage.

Embodiments of the invention provide a system and method for inducing exfoliation of pancreatic cells to yield a sufficient quantity of cells for analysis and detection of abnormal cells. For example, whereas some other prior pancreatic juice sampling typically yielded fewer than 50 pancreatic cells and contained no intact tissue segments, the embodiments described herein consistently yield specimens containing more than 100 pancreatic cells and also capture intact tissue sheets. In some embodiments, tissue morphological analysis is performed. Evaluation of a tissue, optionally, an intact tissue sheet, allows appreciation of the honeycomb pattern of cells that characterizes benign glandular epithelium, whereas detection of effacement of that honeycomb pattern characterizes dysplasia and cancer.

An aspect of some embodiments of the invention relates to non-invasive early detection of prostate cancer and/or dysplasia, by testing an isolated sample of urine and/or ejaculate comprising prostatic cells and/or tissue(s) from a patient's prostate. In some embodiments, the prostate is insonated to generate shear waves. Without being bound to theory, the shear waves partially segment the target epithelium by creating lateral breaks between cells and/or their backing, for example, as described herein optionally, by applying high intensities of focused ultrasound waves.

Exfoliated cells and tissue from the prostate drain into the urethra and exit the body during ejaculation and/or urination and collected for analysis. In addition, the exfoliated cells from urethra the can enter the bladder (e.g., retrograde into the bladder and then passed out with the subject's urine and collected for analysis.

Early detection of prostate cancer potentially increases the chances of treatment success. However, since the disease often has no symptoms until it is advanced, it is frequently detected at advanced stages, when the cancer is debilitating and/or life-threatening. Embodiments of the invention, potentially allow enhanced and/or expedited dislodgement of prostatic cells and/or tissue(s), thereby making urine and/or ejaculate collection a viable and/or non-invasive option for early detection of prostate cancer and pre-cancer. Urine and/or ejaculate collection has the potential advantage of being minimally invasive tests. In some embodiments, urine and/or ejaculate samples collection can be employed for prostatic cancer screening and/or for prostatic cancer surveillance (e.g., monitoring patients who have been diagnosed with prostate cancer but are not undergoing active treatment, or patients with abnormal cells in previously diagnosed samples, or patients diagnosed with prostate cancer that are undergoing treatment for monitoring treatment success and/or the progress of the disease, optionally a sample is collected from the patient at a certain frequency, for example every week or month, optionally, depending on the state of the disease).

Embodiments of the invention provide a system and method for inducing an isolated sample of urine and/or ejaculate comprising exfoliated prostatic cells with sufficient quantity for analysis and detection of abnormal cells. In embodiments of the invention, the cells are exfoliated from the prostate in a greater quantity and/or rate and comprise cell clumps compared to a non-insonated prostate and/or compared to a prostate not insonated with relatively high intensities of focused ultrasound waves to generate shear waves.

Some methods and systems described herein are used for obtaining a sample comprising at least one of any of cells, contiguous cells, cell sheets, cell group(s) and/or cluster(s), and/or at least one tissue fragment exfoliated from a prostate. In some embodiments, the sample comprises intact tissue sheet(s) and/or intact tissue fragment(s).

Insonating the prostate to generate shear waves potentially allows to reduce and/or avoid microbubbles insonation mechanic effect outside the prostate and/or in the prostate. This has a potential advantage of reducing and/or avoiding undesired interaction of the microbubbles with the surrounding, organs nerves, and/or blood vessels. For example, reducing and/or avoiding the risk of affecting the bladder, and the urethra and/or causing impotence, for example, by the microbubbles. In addition, insonating the prostate to generate shear waves potentially allows focusing the ultrasound waves at the target tissue/organ, having the potential advantage of reducing and/or avoiding the risk of interacting with surrounding organs nerves, and/or blood vessels by the ultrasound waves.

In some embodiments, prostate stimulation is performed to potentially promote prostate fluid secretion. In some embodiments, the stimulation comprises sexual stimulation, prostate massage, and/or electroejaculation (e.g., providing the prostate with an electric current).

In embodiments of the invention, patient urination is promoted. In some embodiments, at least one diuretic substance, such as Furosemide or a similar diuretic may be administered to a patient to increase urine output. Alternatively or additionally, the patient is prompted to drink before, after, and/or during the ultrasound treatment.

### Preserving Samples

Once a sample has been collected, the cell sample may optionally be kept under conditions and/or treated so as to minimize autolysis.

By way of a non-limiting example, the cell sample may optionally be kept by addition of formalin.

By way of a non-limiting example, when a pancreatic juice sample contains tissue from a pancreas, and the sample is bathing in pancreatic enzymes there may be digestion of the cells of the pancreatic sample in the pancreatic juice competing with cross linkage fixation of the same cells that is being performed by formalin.

Enzymatic action is inhibited by cold. In some embodiments the cell sample is optionally kept in a jar on ice or embedded in ice.

By way of a non-limiting example, when a sample contains tissue from a pancreas, and the sample is bathing in pancreatic enzymes there may be catalytic action on the cells of the pancreatic sample in the pancreatic juice competing with cross linkage fixation that is being performed by formalin. Pancreatic enzymes require a relatively alkaline environment and are disabled by an acidic environment.

By way of a non-limiting example, the pH of the formalin or other fixative preservative into which the fluid sample will placed for fixation is optionally lowered.

By way of a non-limiting examples, the pH lowered fixative could be formalin, alcohol or a mixture of the two.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

Before explaining at least one embodiment of the disclosure in detail, it is to be understood that the disclosure is not necessarily limited in its application to the details set forth in the following description or exemplified by the Examples. The disclosure is capable of other embodiments or of being practiced or carried out in various ways.

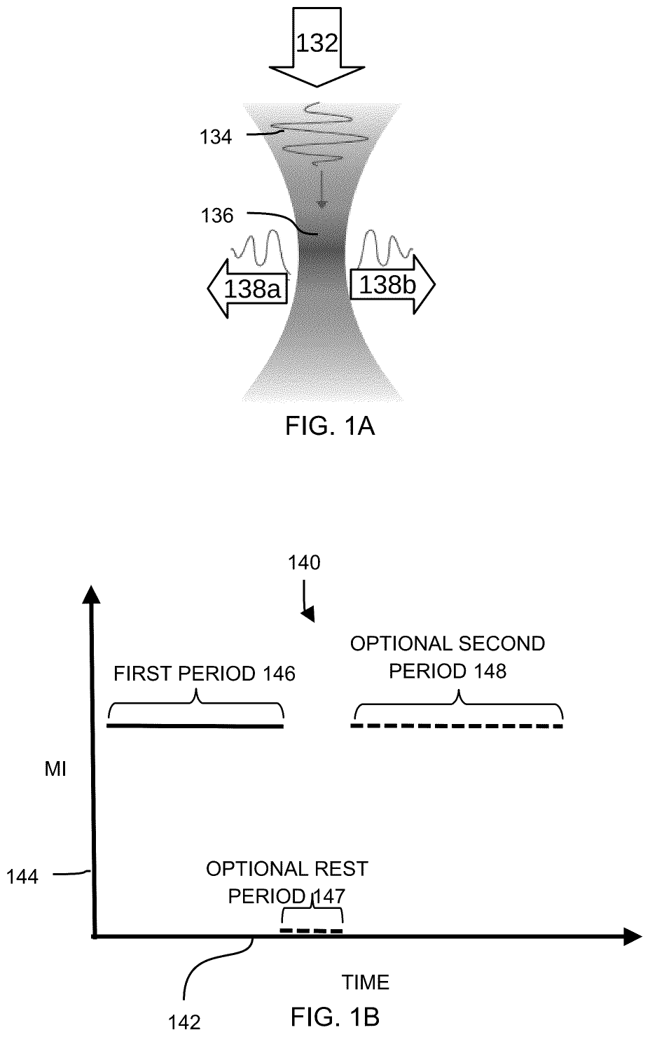

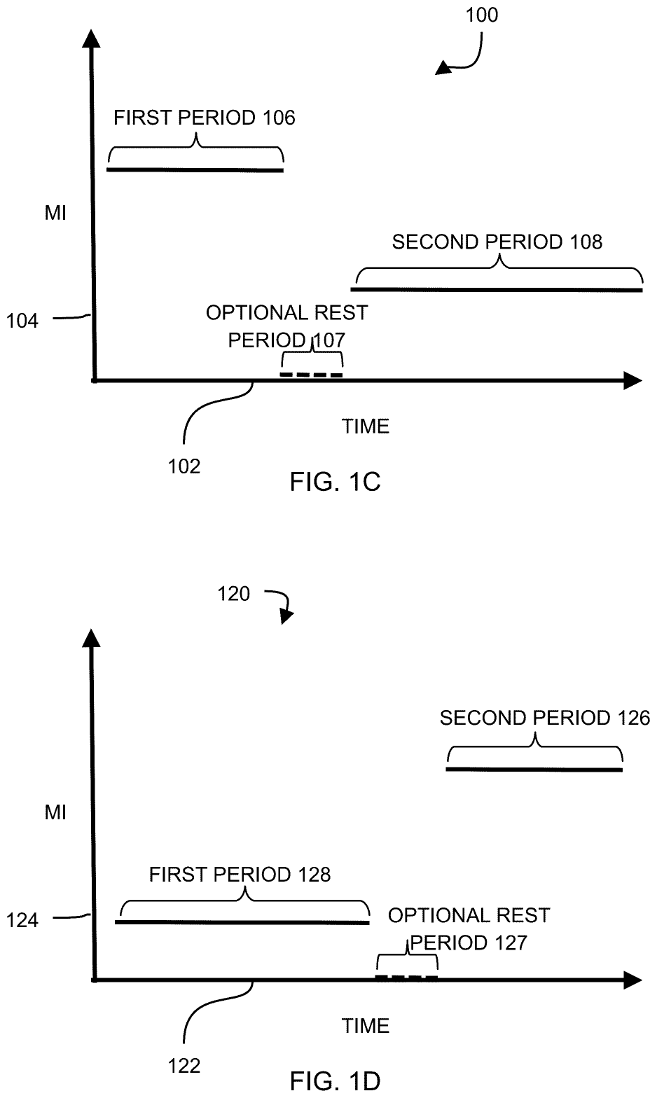

Reference is now made to FIG. 1A, which is a simplified qualitative illustration of insonation which is used to cause exfoliation of cytopathologic cell samples according to an example embodiment of the invention.

FIG. 1A shows an ultrasound source 132 producing an ultrasound wave 134 into tissue.

FIG. 1A shows an acoustic radiation force field from a focused ultrasound beam 134. The force field, or region of excitation, lies within a geometric shadow of a transmit aperture and is typically, most energetic near the focal point 136. A high intensity focused ultrasound beam 134 can be used to push on tissue, to generate shear waves 138a 138b, which propagate laterally away from the region of excitation, or focal point 136.

In some embodiments, a focused ultrasound beam is optionally used to induce exfoliation of cells and molecules from a targeted tissue, such as, by way of a non-limiting example, epithelium. by application of a focused ultrasound beam.

In some embodiments, the focused ultrasound beam is produced to induce a mechanical index (MI) in a range between 0.4 and 1.8.

In some embodiments, the focused ultrasound beam is arranged to produce a beam width at focus in a range between 2 mm and 10 mm.

In some embodiments, the ultrasound beam is produced with a pulse width in a range between 100 microseconds and 800 microseconds.

In some embodiments, the ultrasound beam is produced with a pulse repetition frequency in a range between 4 Hz and 20 Hz.

Reference is now made to FIG. 1B, which is a simplified qualitative illustration of insonation during a procedure for producing cytopathologic cell samples according to an example embodiment of the invention.

FIG. 1B shows a graph 140 of insonation levels over time. The graph 140 has an X axis 142 of time, and a Y axis 144 of mechanical index (MI) in a target tissue, which corresponds to a level of insonation.

FIG. 1B shows a first period 146 of insonation at a relatively high level of insonation, by way of a non-limiting example at a level of insonation produced to induce a mechanical index (MI) in a range between 0.4 and 1.8.

In some embodiments, an optional rest period 147 may be present between the first period 146 and an optional second period 148.

Reference is now made to FIG. 1C, which is a simplified qualitative illustration of levels of insonation during a procedure for producing cytopathologic cell samples according to an example embodiment of the invention.

FIG. 1C shows a graph 100 of insonation levels over time. The graph 100 has an X axis 102 of time, and a Y axis 104 of mechanical index (MI) in a target tissue, which corresponds to a level of insonation.

FIG. 1C shows a first period 106 of insonation at a higher level of insonation, and a second period 108 of insonation at a lower level of insonation, as described herein.

In some embodiments, an optional rest period 107 may be present between the first period 106 and the second period 108.

Reference is now made to FIG. 1D, which is a simplified qualitative illustration of levels of insonation during a procedure for producing cytopathologic cell samples according to an example embodiment of the invention.

FIG. 1D shows a graph 120 of insonation levels over time. The graph 120 has an X axis 122 of time, and a Y axis 124 of mechanical index (MI) in a target tissue, which corresponds to a level of insonation.

FIG. 1D shows a first period 126 of insonation at a lower level of insonation, and a second period 128 of insonation at a higher level of insonation, as described herein.

In some embodiments, an optional rest period 127 may be present between the first period 126 and the second period 128.

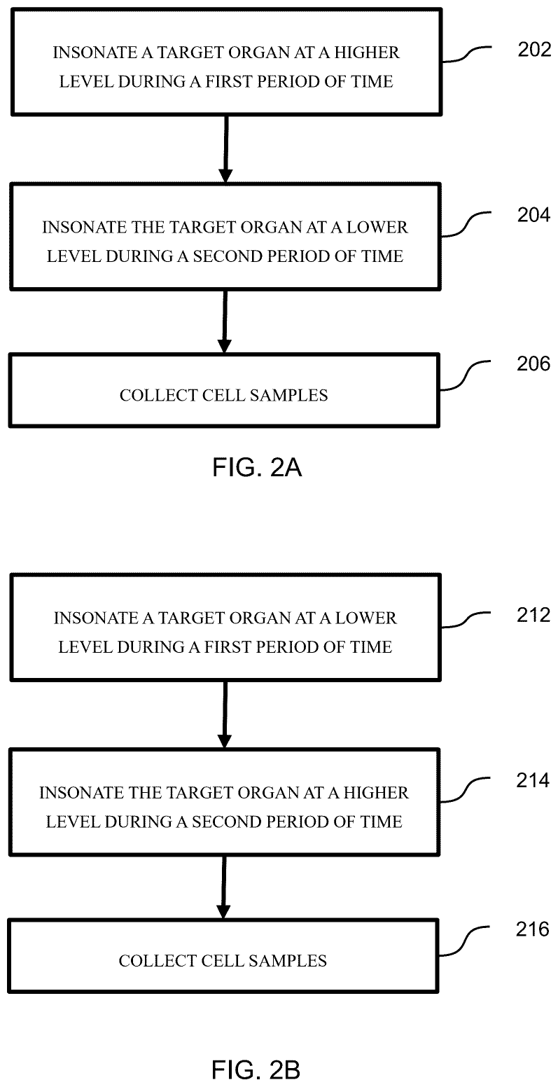

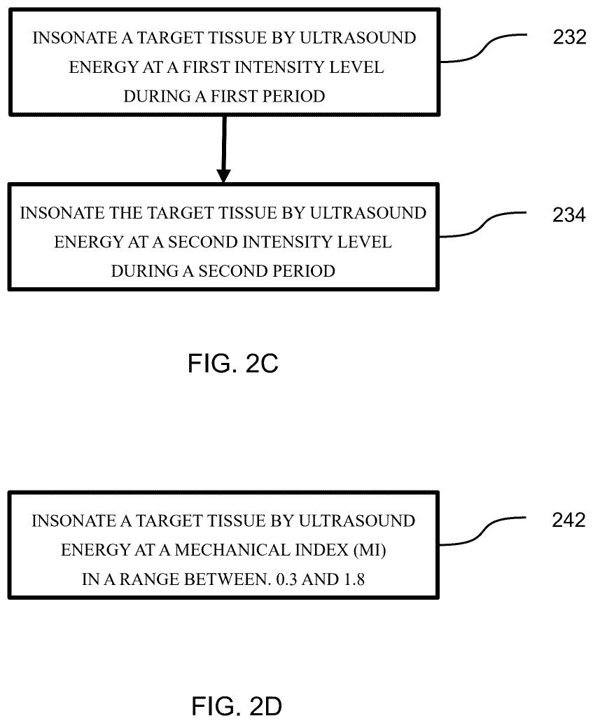

Reference is now made to FIG. 2A, which is a simplified flow chart illustration of a method for producing cytopathologic cell samples according to an example embodiment of the invention.

The method of FIG. 2A includes:
insonating a target organ at a higher level during a first period of time (202);
insonating the target organ at a lower level during a second period of time (204); and
collecting cell samples (206).

In some embodiments, there is an optional period of time, not shown, between the first period of time of insonation at the higher level, and the second period of time of insonation at the lower level, during which there is no insonation.

Reference is now made to FIG. 2B, which is a simplified flow chart illustration of a method for producing cytopathologic cell samples according to an example embodiment of the invention.

The method of FIG. 2B includes:
insonating a target organ at a higher level during a first period of time (222);
insonating the target organ at a lower level during a second period of time (214); and
collecting cell samples (216).

In some embodiments, there is an optional period of time, not shown, between the first period of time of insonation at the lower level, and the second period of time of insonation at the higher level, during which there is no insonation.

Reference is now made to FIG. 2C, which is a simplified flow chart illustration of a method for producing cytopathologic cell samples according to an example embodiment of the invention.

The method of FIG. 2C includes:
insonating a target tissue by ultrasound energy at a first intensity level during a first period (222);
insonating the target tissue by ultrasound energy at a second intensity level during a second period (224);
wherein
the first intensity level is higher than the second intensity level.

Reference is now made to FIG. 2D, which is a simplified flow chart illustration of a method for inducing exfoliation according to an example embodiment of the invention.

The method of FIG. 2D includes insonating a target tissue by ultrasound energy at a Mechanical Index (MI) in a range between 0.3 and 1.8 (242).

In some embodiments, the insonating the target tissue is by a focused ultrasound beam.

In some embodiments, the insonating is performed by repeating ultrasound pulses of a duration between 20 μS and 800 μS.

In some embodiments, the insonating is performed by repeating ultrasound pulses at a rate in a range between 1 to 1400 times per second.

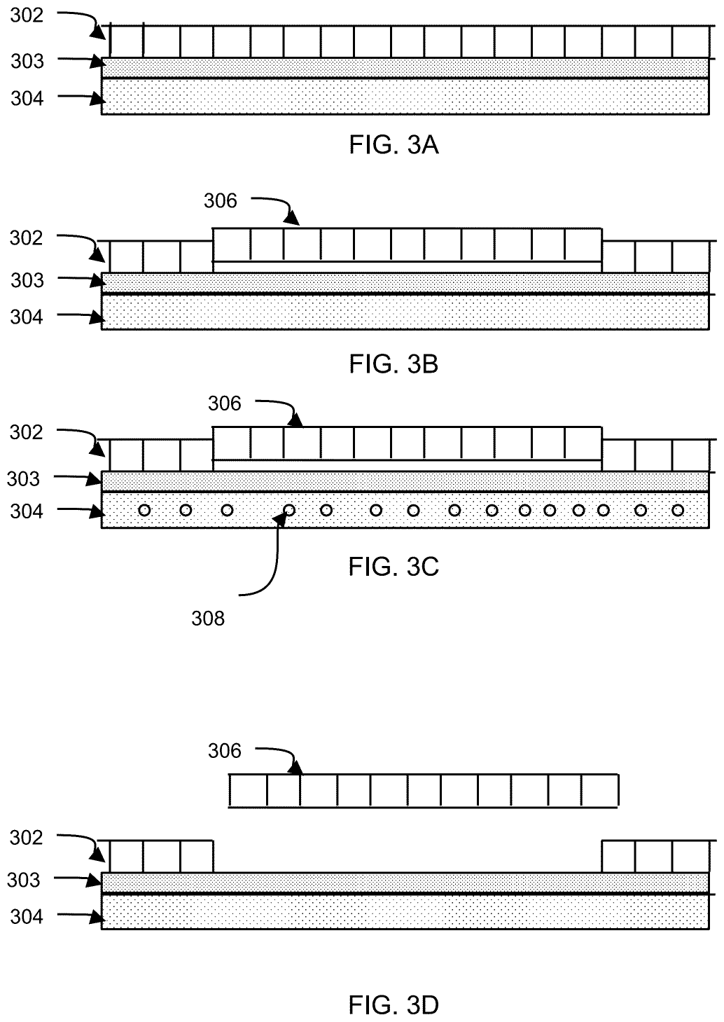

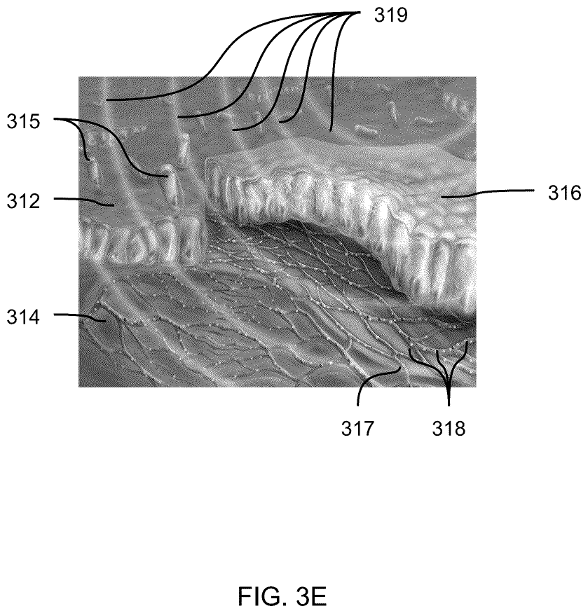

Reference is now made to FIGS. 3A-3E, which are simplified illustrations of using ultrasound and ultrasound contrast agent to produce a cell sample according to an example embodiment of the invention.

FIG. 3A shows an example of three layers of tissue. A first layer of epithelium 302, a second layer of basement membrane (lamina propria) 303, and a third layer of submucosa 304 of cells in a target organ, attached to each other both horizontally and vertically. It is noted that in the third layer of submucosa 304 there are capillaries.

FIG. 3B shows the three layers 302 303 304 of cells following insonation, where a group of epithelium cells 306 has separated from the other cells.

In some embodiments, the separation is achieved by eliciting shear waves by insonation.

In some embodiments, the group of cells 306 may be completely separated, and may flow along a pancreatic duct and be collected as a cell sample.

In some embodiments, additional insonation, optionally at lower energy level(s) than used to produce the shear waves, is used to complete a separation of the group of cells 306.

In some embodiments, ultrasound contrast agent material, such as secretin, is provided to the target organ, and the target organ is further insonated, optionally at lower energy level(s) than used to produce the shear waves.

FIG. 3C shows the three layers 302 303 304 of cells, the group of cells 306, and bubbles 308 formed in capillaries in the third layer of submucosa 304 by insonating the ultrasound contrast agent. The bubbles may further act to separate the group of cells 306 from their neighboring cells.

FIG. 3D shows the group of cells 306 separated from the neighboring cells, which potentially enables the group of cells 306 to flow with surrounding fluid and optionally be collected in a cell sample.

By way of a non-limiting example, one cell layer 302 may be an epithelial layer in a pancreatic duct, and the group of cells 306 may be a group of epithelial cells separated from the pancreatic tissue, to flow along a pancreatic duct and optionally be collected as a cell sample.

Reference is now made to FIG. 3E, which is a simplified illustration of using ultrasound and ultrasound contrast agent to produce a cell sample according to an example embodiment of the invention.

FIG. 3E shows epithelium 312 316, a basement membrane 314, and blood vessels 317, being insonated 319.

Describing FIG. 3E in terms of a pancreas, FIG. 3E shows pancreatic ductal epithelium 312 316 as a single layer of columnar cells 312 315 316 sitting on top of a basement membrane 314. A shear wave produced by ultrasound insonation breaks lateral bonds between the columnar cells 312 315 316 and a subsequent wide beam insonation 319 of microbubbles 318 breaks bonds between basal bands of the columnar cells 315 316 and the basement membrane 314 that is below them. The insonation 319 does not break basal bonds between one layer of glandular cells and another layer of glandular cells below them.

### Various Example Embodiments

In some embodiments, ultrasound energy absorbed within target tissue, by way of a non-limiting example within a pancreas, causes mechanical vibrations that enhances pancreatic ductal cells and, optionally, tissue fragments to disassociate and/or exfoliate. A patient, in the case of obtaining a pancreas sample, is subsequently optionally injected with secretin, a material that induces pancreatic secretion. Some of the exfoliated and dislodged cells and tissue fragments may be deposited into the pancreatic juice, which is then collected, for example endoscopically.

In some embodiments, the cells and/or tissue fragments in cell-enriched samples obtained are then analyzed morphologically and/or using molecular biomarkers to detect a presence or absence of cellular abnormality.

A non-limiting example method of obtaining cell samples and tissue fragments from an organ of a subject may include: insonating the organ with an amount of a narrow beam higher intensity ultrasonic energy effective to elicit shock waves and/or shear waves causing an associated vibration of the tissue or organ, followed by insonating the organ of the subject with a wide beam, lower intensity longer pulse width ultrasound while the subject has been injected with ultrasound contrast agent, so as to thereby elicit exfoliation of cells or an epithelia tissue fragment from the organ of the subject.

Such methods can potentially increase a total number of cells to be expressed in the pancreatic juice. Moreover, the procedures set forth herein may induce the separation of intact tissue fragments from the pancreas.

The methods, when applied to the other organs or body sites, such as, the mediastinum, pleura, pericardium, peritoneum, lung, breast, salivary glands, meninges, pancreatic ducts, pancreatic cysts, kidney, liver, prostate, bladder, or ovaries, potentially increase a total number of cells expressed in the surrounding fluids (e.g., fluids found in and/or near the organ and/or to which the organ drains). Additionally, the methods set forth herein potentially induce separation of intact tissue fragments from the above-cited exemplary organs. For example, the methods set forth herein are utilized to induce exfoliation of lung cells into surrounding sputum or to induce exfoliation of bladder cells and prostate and/or bladder tissue into surrounding urine.

In some embodiments, a method is provided for obtaining cell samples and tissue fragments from an organ of a subject including: insonating a target organ of the subject with an amount of ultrasonic energy effective to elicit shear waves, potentially causing an associated vibration of the target tissue or organ.

In some embodiments, a method is provided for obtaining cell samples and tissue fragments from an organ of a subject including: insonating a target organ of the subject with an amount of ultrasonic energy effective to elicit shear waves, potentially causing an associated vibration of the target tissue or organ, followed by insonating the target organ of the subject with wide beam, low intensity long pulses ultrasound after the subject has been injected with ultrasound contrast agent, so as to thereby elicit exfoliation of cells or epithelial tissue fragment(s) from the target organ.

In some embodiments, a method is provided for obtaining pancreatic cells from a subject including: insonating a pancreas of the subject with an amount of single frequency ultrasonic high energy pulse, of 2-40 μs in duration, effective to elicit exfoliation of cells into a duct of a pancreas in a subject.

In some embodiments, the ultrasound waves are emitted at a frequency of 3 MHz.

In some embodiments, the ultrasound waves are emitted at a different frequency in a range associated with ultrasound.

In some embodiments, a method is provided for obtaining pancreatic cells from a subject comprising: insonating a pancreas of the subject with an amount of ultrasonic energy from a multi-frequency array effective to achieve an asymmetric ultrasound wave, mostly positive, or mostly negative immediately followed by mostly positive, at a predetermined point in the pancreas and elicit exfoliation of cells into a duct of a pancreas in the subject. Referring to mostly positive and mostly negative means referring to pulses which are obtained by constructive interference of 2 or more ultrasound waves, optionally at integer multiples of their fundamental transmitted frequency, for example at a fundamental transmitted frequency f, at 2f, at 3f and so on, while phases between the transmitted frequencies are designed to produce a summation of all positive (or negative) amplitudes at a specific depth within the tissue.

In some embodiments, a method is provided of treating a subject for a pancreatic disorder including:
a) determining if the subject has pancreatic dysplastic cells or pancreatic cancer cells in their pancreas by the method described herein; and
b) effecting chemotherapy, radiotherapy, immunotherapy or a pancreatectomy in a subject found in a) to have pancreatic dysplastic cells or pancreatic cancer cells in their pancreas.

In some embodiments, the determining if the subject has pancreatic dysplastic cells or pancreatic cancer cells in their pancreas is effected through one or more of cellular morphological analysis, tissue morphological analysis, or molecular marker analysis.

In some embodiments, a method is provided for increasing the efficacy of cell sample collection from a tissue or an organ, in an assay procedure on a subject including, prior to collecting a cell sample, insonating the tissue or organ of the subject with an amount of ultrasonic energy effective to elicit shear wave(s) within the tissue or organ, so as to thereby elicit exfoliation of cells or tissue fragments from the tissue or organ followed by collecting the cell sample.

In some embodiments, a method is provided for performing an assay on a sample of cells or tissue from a subject so as to determine if the cells or tissue comprise cancerous or precancerous cells or tissue, including:
a) receiving a sample of cells or tissue, where the sample has been previously obtained by a method of creating shear waves within the tissue and organ of the subject, so as to thereby elicit exfoliation of cells or an epithelia tissue fragment from the organ in a subject, and collecting a sample of the exfoliated cells or tissue;
b) performing one or more of cellular morphological analysis, tissue morphological analysis, and molecular marker analysis, so as to determine if the cells or tissue comprise cancerous or precancerous cells or tissue.

In some embodiments, a method is provided for early detection of dysplastic and cancerous cells in a pancreas including application of ultrasound energy directed to the pancreas to induce cellular exfoliation, followed by endoscopic collection of pancreatic fluid containing exfoliated cells and/or cell clusters, for molecular examination and microscopic morphological examination.

In some embodiments, there is provided a sample of a body fluid, wherein the sample has been directly obtained from a subject who has had a tissue or organ insonated, wherein the sample comprises epithelial or other cells from the tissue or organ at a level enriched by more than 2 times relative to a level of epithelial or other cells in an otherwise identical sample obtained from a subject who has not had the tissue or organ insonated.

In some embodiments, a method is provided for obtaining a sample of contiguous pancreatic duct cells from a subject including:
administering to the subject, within one hour before or after initiation of insonation with ultrasonic energy, an amount of secretin polypeptide, effective to elicit pancreatic secretion;
insonating a pancreas of the subject with an amount of ultrasonic energy at a predetermined point in the pancreas to elicit exfoliation of cells into a duct of a pancreas in the subject; and
removing a sample of fluid containing contiguous pancreatic duct cells from the subject.

In some embodiments of the invention, methods and/or systems described herein are employed to induce exfoliation of prostatic cells.

Reference is now made to FIG. 4A, which is a front perspective view of an ultrasound probe according to an embodiment of the invention.

FIG. 4A shows a front view of an ultrasound transducer or probe 410. The probe 410 is formed of one or more piezoelectric elements housed within an outer casing 412. In some embodiments of the invention the outer casing 412 has a strap attached thereto 414, to attach the probe to a patient.

Reference is now made to FIG. 4B, which is a schematic view of a probe configuration according to an example embodiment of the invention.

FIG. 4B shows the outer casing 410 of FIG. 4A, and an arrangement of one or more ultrasound piezoelectric elements 422 within the outer casing 410.

In some embodiments, one or more optional receiver elements 424 may optionally be arranged in the outer casing 410. In some embodiments, the receiver elements 424 are optionally cavitation detectors.

In some embodiments, the probe 412 is optionally programmed to produce an unfocused beam of ultrasound energy.

In some embodiments, one or more of the ultrasound transducers 422 in the probe 412 are optionally programmed to produce a focused beam of ultrasound energy.

In some embodiments, several ultrasound transducers 422 in the probe 412 are optionally programmed to produce together a focused beam of ultrasound energy.

It will be understood by those of ordinary skill in the art that any number of piezoelectric elements may be used in different embodiments of the invention.

In some embodiments of the invention, one or more cavitation detectors 424 are optionally provided on the probe to detect the incidence of unstable cavitation. The cavitation detectors 424 may comprise passive cavitation detectors (PCD) or another type of hydrophone.

Reference is now made to FIG. 4C, which is a simplified drawing of an array of elements in an ultrasound transducer according to an example embodiment of the invention.

FIG. 4C is intended to show a one-dimensional array 431 of elongated transducer units 432.

In some embodiments, an ultrasound probe or transducer is formed of multiple transducer crystals, or of a single crystal that is etched or similarly partitioned to form a series of thin, uniformly arranged transducer units 432. The transducer units may be arranged on a flat, a curved, or a concave foundation.

In some embodiments, the probe is configured to deliver either a non-focused homogenous beam, or a steerable point-focused or steerable bar-shaped focused beam, at sufficient radiation intensities to a target organ such as a pancreas in order to impart sufficient energy to the tissue of the target organ, and in some embodiments to a contrast agent flowing within its vasculature, to induce exfoliation of cells and/or tissue.

In some embodiments a transducer is composed of an array 431 of crystal elements 432 that are long, by way of a non-limiting example on the order of 120 mm, and narrow, by way of a non-limiting example 4 mm, as shown in FIG. 4C. Excitation of a subset of such elements may result in a bar-shaped focused steerable beam with an approximate dimension at the focal plane of 3 mm×10 mm.

In some embodiments a number of the transducer crystal elements 431 may be in a range between 2 and 10 or even up to a range between 2 and 100. In some embodiments the number of transducer crystal elements 431 is odd, enabling defining a center transducer crystal element and a symmetrical distribution of transducer crystal elements partitions on either side of the center transducer crystal element.

Reference is now made to FIG. 4D, which is a simplified drawing of an array of elements in an ultrasound transducer according to an example embodiment of the invention.

FIG. 4D is intended to show a two-dimensional array 435 of transducer units 436.

In some embodiments the transducer crystal elements 431 shown in FIG. 4C are etched into partitions along a perpendicular axis as shown in FIG. 4D. by way of a non-limiting example, the number of partitions may be in a range between 2 and 10 or even up to a range between 2 and 100. In some embodiments the number of partitions is odd, enabling defining a center partition and a symmetrical distribution of partitions on either side of the center partition.

Excitation of a such elements, or of a subset of such elements, may result in a point-focused steerable beam.

In some embodiments, the focus of the beam may have an approximate dimension at the focal plane of 2 mm×4 mm.

In embodiments the ultrasound probe is configured to insonate a combination, sequentially or simultaneously, of: 1) long pulses of point-focused or bar-shaped focused steerable beams; 2) long pulses of substantially homogenous flat beams covering an entire area, such as an organ or an area of an organ, substantially simultaneously.

In some embodiments the probe, the 2D array of transducer crystals, or the single crystal etched into columns and rows, which enables to insonate sequentially or simultaneously a combination of long pulses of point-focused or bar-shaped steerable beams, and long pulses of a significantly homogenously flat beam, also allows imaging in 3D with lateral and elevation focusing.

In some embodiments the probe, the 2D array of transducer crystals, or the single crystal etched into columns and/or rows, enables reduced peak-excitation intensities and better power management per crystal element, and heat control of the probe relative to a larger transducer crystal and/or to a non-etched large transducer crystal.

In some embodiments the probe includes a plurality of cavitation detectors and an associated computer system that is configured to i) detect an incidence of inertial cavitation; ii) determine a plane at which an incidence of inertial cavitation is detected; iii) determine if the plane wherein such cavitation is detected is located within a target organ; and iv) reduce the intensity of ultrasound energy if the plane where the inertial cavitation is detected is within a plane identified as being within the target organ.

In some embodiments, a system is provided, including a probe cable and connector(s), a belt, an electronic module, a control unit, a processing unit and a Graphics User Interface (GUI).

In some embodiments, the system may optionally include a disposable sheath that envelopes the probe and/or its cable, or at least some of the probe and/or cable, so as to allow use of the system to be sterile, while allowing attachment to a belt that holds the probe in place.

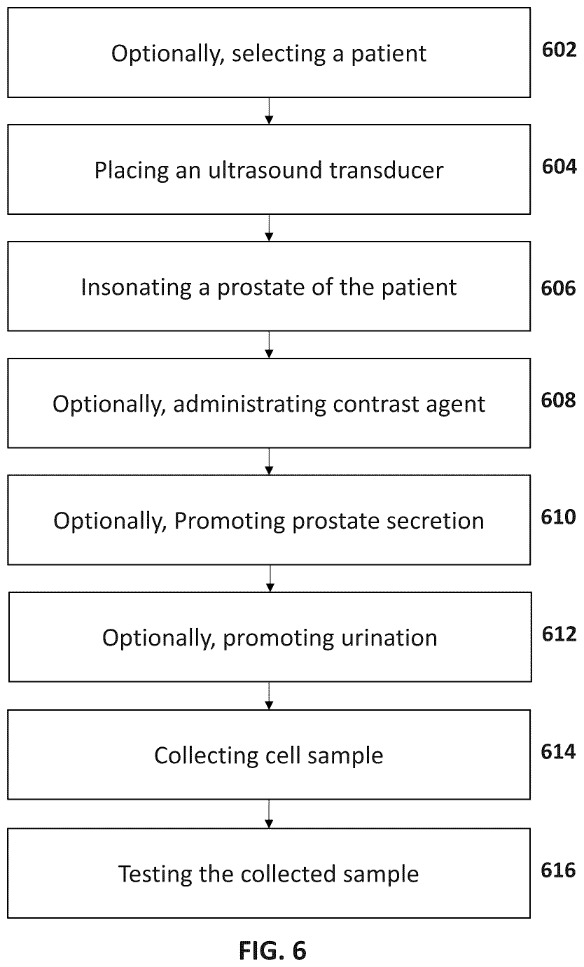

Reference is now made to FIG. 6, which is a flow chart of a method for producing cytopathologic cell samples from a prostate, according to an example embodiment of the invention. In some embodiments, methods and/or systems described in this document are employed to induce exfoliation of prostatic cells and/or tissue(s), mutatis mutandis. The exfoliated cells and/or tissue are drained into the urethra and exit the body during ejaculation and/or enter the bladder. Such cells are then passed out with the subject's ejaculate and/or urine and collected for analysis, potentially allowing early detection of prostate cancer and pre-cancer. Urine and/or ejaculation collection is non-invasive and/or minimally invasive procedure, having the potential advantage of reducing patient discomfort and/or pain and increasing compliance with prostate cancer testing. It will be understood that the step of administering secretin or the like is optionally omitted in this embodiment of the invention.

The method of FIG. 6 includes:

Optionally, selecting a patient (602). Prostate cancer is the most common cancer in America and is a disease that could be debilitating or fatal if not detected early. However, early detection potentially increases the chances of treatment success. Since prostate cancer often has no symptoms until the disease is advanced, the method can be used for prostate cancer screening. Alternatively or additionally to prostate cancer screening, the method can be used for prostate cancer surveillance. For example, surveilling patients diagnosed with prostate cancer, and optionally not undergoing active treatment. Another example is surveilling patients with former results of abnormalities in the collected sample.

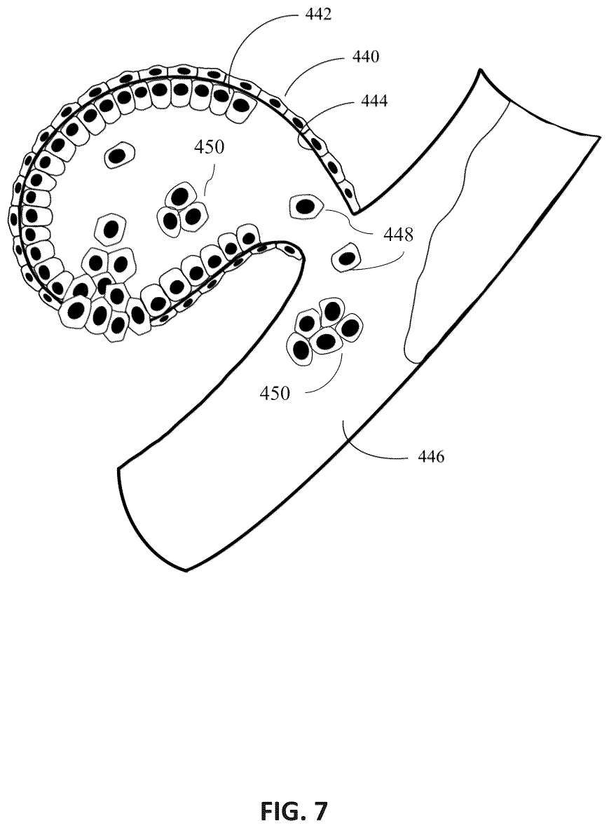

Placing an ultrasound probe (e.g., ultrasound transducer) (604), optionally in proximity to the prostate, as shown for example in FIG. 7. The proximity to the prostate has the potential advantage of reducing and/or avoiding damaging surrounding blood vessels and/or nerves, impairing the bladder and/or causing impotence. In some embodiments, the probe is introduced to a predetermined point near the prostate, optionally, introduced thereto rectally.

Insonating the patient's prostate (606), with an amount of ultrasonic energy at a predetermined point in the prostate to elicit exfoliation of cells, optionally, into the urethra of the subject. In some embodiments, the ultrasound energy is at a higher intensity during a period.

As described above, in some embodiments of the invention, shear waves are generated by high intensity, long duration (e.g., 100 μs-800 μs) pulses of focused ultrasound beams. For example, pulse duration of about 100 μs-800 μs, or 50 μs-100 μs, or 150 μs-300 μs, or about 200 μs, or 250 μs, or 180 μs, or lower or higher or intermediate numbers of microseconds. Typically, such ultrasound beams are perpendicular to the surface of a prostatic epithelium. For example, the ultrasound beams are between about 60-120 degrees, relative to the rectum and/or perineum, or about 75-105 degrees, or about 80-100 degrees, or about 85, or about 95, or lower or higher or intermediate numbers of degrees. The shear-wave-inducing ultrasound beams traverse through the prostate and produce pressure changes that cause fast expansions and contractions that are typically parallel to a face plane of the tissue. These mechanical changes potentially produce separation among lateral connections between epithelial cells. It is noted that standard imaging ultrasound uses a pulse width of 2 μs. In some embodiments of the invention, the ultrasound energy at a higher intensity during a first period, optionally followed by ultrasound energy at a lower energy during a second period. There may or may not be a rest period between the first period of insonation and the second period of insonation. The second period potentially lifts the partially segmented epithelium from the organ surface, to induce exfoliation of cells and tissue fragments.

In some examples, the first period of insonation lasts in a range between 1 and 15 minutes of shear waves at higher MI. For example, between 0.5 and 11 minutes, between 10 and 20 minutes, between 5 and 30 minutes, or about 5, or 12, or 17, or lower or higher or intermediate numbers of minutes. In some examples, the second period of insonation lasts in a range between 10 and 20 minutes. In some examples, the second period of insonation lasts in a range between 3 and 30 minutes. For example, between 1 and 15 minutes, between 8 and 35 minutes, between 15 and 45 minutes, or about 9, or 20, or 25, or lower or higher or intermediate numbers of minutes.

Alternatively or additionally, insonating the prostate with lower intensities of ultrasound waves is performed prior to applying the higher intensity ultrasound. In other embodiments, insonating the patient's prostate can be performed according to other methods, for example, as described in patent application U.S. Ser. No. 17/367,658 and/or U.S. Ser. No. 17/028,588. In some embodiments, this method might have a disadvantage for treating the prostate, since that relatively increased amount of microbubbles and/or elongated duration of microbubbles insonation may cause undesired interaction with the surroundings of the prostate, such as blood cells, nerves, and organs, for example, the bladder and the urethra.

Optionally, administrating the patient an ultrasound contrast agent (908). In some embodiments, the contrast agent is administered subsequent to the first period of insonation (e.g., insonation with higher intensities). Contrast agent which may be 1μ-4μ sized gas microbubbles, optionally encapsulated by protein, lipid or a polymeric shell, expand and contract due to the changes in their local pressure changes when exposed to ultrasound waves. Thus, when injected intravenously to a subject, the microbubbles flow through the vascular system and all its capillaries, and reach the rich microvasculature surrounding the epithelial cells of the prostate. In some embodiments, co-employment of an ultrasound beam aimed at the prostate, and the existence of microbubbles within its microvasculature, results in exfoliation of cells and/or tissue fragments. Without being bound to theory, this may cause synchronized expansion-contraction of the microvasculature that underlies the epithelial layer, thus dislocating cells and clusters of cells by disruption of cell junctions between the base of an epithelial cell and basement membrane below 20) it. In some embodiments, after introducing the microbubbles the prostate is subjected to wide-area ultrasound energy. The ultrasound application of embodiments of the invention may be described as Low Intensity Non-Focused Ultrasound (LINFU application®). In some embodiments, the first period of insonation to generate shear waves, optionally at higher MI lasts for ranges between 1 to 15 minutes, optionally, about 5 minutes. For example, between 0.5 and 11 minutes, between 10 and 20 minutes, between 5 and 30 minutes, or about 5, or 12, or 17, or lower or higher or intermediate numbers of minutes. The first period is followed by a second period of insonation of microbubbles at lower MI. that lasts for approximately 10-20 minutes. For example, between 5 and 12 minutes, between 8 and 25 minutes, between 15 and 30 minutes, or about 6, or 22, or 25, or lower or higher or intermediate numbers of minutes.

Optionally, promoting prostate fluid secretion (910). Promoting prostate fluid secretion potentially allows obtaining relatively quickly the exfoliated cells and/or tissue fragment(s) shed from the prostate, having the potential advantage of reducing the time required for collecting a sample and/or a required sample volume (e.g., volume of prostatic fluid). In some embodiments, said promotion secretion comprises promoting ejaculation, for example by sexual arousal. In some embodiments, said promotion secretion comprises stimulating the prostate, having the potential advantage of allowing the promotion of prostate fluid secretion for patients who suffer from impotence and/or are unable to ejaculate for other reasons, for example, as a result of a spinal cord injury or other neurological conditions.

In some embodiments, said stimulation comprises electroejaculation, which includes sending an electric current to the prostate gland. Optionally, the electrical current causes the prostate gland and seminal vesicles to contract and release prostatic secretion (e.g., ejaculate). Alternatively or additionally, the stimulation comprises pressing the prostate gland, for example, during a rectal examination and/or prostate massage.

Optionally, promoting urination of the patient (912), since exfoliated epithelial cells and/or tissue(s) can be drained into the bladder and subsequently be expelled in urine. Promoting urination has a potential advantage of allowing to obtain relatively quickly the exfoliated cells and/or tissue fragment(s) shed from the prostate, having the potential advantage of reducing the time required for collecting a sample and/or a required sample volume (e.g., volume of urine). In some embodiments, said prompting urination comprises administrating the patient with at least one diuretic substance, such as Furosemide. Alternatively or additionally, the patient is prompted to consume fluids before, after, and/or during the ultrasound treatment. Optionally, the prostate is insonated when the patient's bladder is full and/or nearly full.

In some embodiments, a sterile urine sample is obtained by catheterization (e.g., connecting a catheter to the patient's bladder for urine collection), during and/or after insonating the prostate. Catheterization has the potential advantage of reducing and/or avoiding sample losses and/or contamination during urine collection by the patient;

Collecting the sample (904). The sample comprises exfoliated cells and/or tissue fragment(s) in one or both of a collected urine and a collected ejaculate (e.g., prostate secretion). The sample comprises at least one of: any of cells, contiguous cells, cell sheets, cell group(s) and/or cell cluster(s) and/or at least one tissue fragment exfoliated from a prostate. In some embodiments of the invention, the cells (e.g., any of cells, cell group(s)/cluster(s), cell sheet(s) and/or tissue fragment(s)) are exfoliated from the prostate in a greater quantity compared to non-insonated prostate and/or compared to a prostate not insonated with relatively high intensities of focused ultrasound waves to generate shear waves. In some embodiments the sample is obtained from the prostate at a greater rate, that is, the number of samples per time unit compared to a non-insonated prostate and/or compared to a prostate not insonated with relatively high intensities of focused ultrasound waves to generate shear waves. In some embodiments of the invention, the method is used for obtaining cell sheets, and/or cell groups and/or tissue fragment samples containing a larger number of cells in the sheet/group/fragment compared to a non-insonated prostate and/or compared to a prostate not insonated with relatively high intensities of focused ultrasound waves to generate shear waves; and

Testing the collected sample (916). In some embodiments, the testing comprises processing the sample to isolate epithelial cells and/or to concentrate epithelial cells in the sample (for example by using cytocentrifuge also referred to as “cytospin”). In embodiments of the invention, the testing comprises analyzing the sample, optionally, by a pathologist. In some embodiments, the testing comprises performing a cytopathologic examination of the cells and/or tissue fragment(s) from the sample.

In some embodiments, cellular morphological analyses, tissue morphological analyses, molecular biological analyses, and molecular biomarkers are used to detect a presence or absence of cellular abnormality (e.g., identifying if the cells as cancerous or dysplastic). In some embodiments, tissue morphological analysis is performed. Evaluation of tissue fragment, optionally, an intact tissue fragment allows appreciation of the honeycomb pattern of cells that characterizes normal epithelium and the contrasting effacement of the honeycomb that occurs in dysplasia and cancer. This evaluation potentially allows to differentiate between inflammatory and dysplastic cellular atypia.

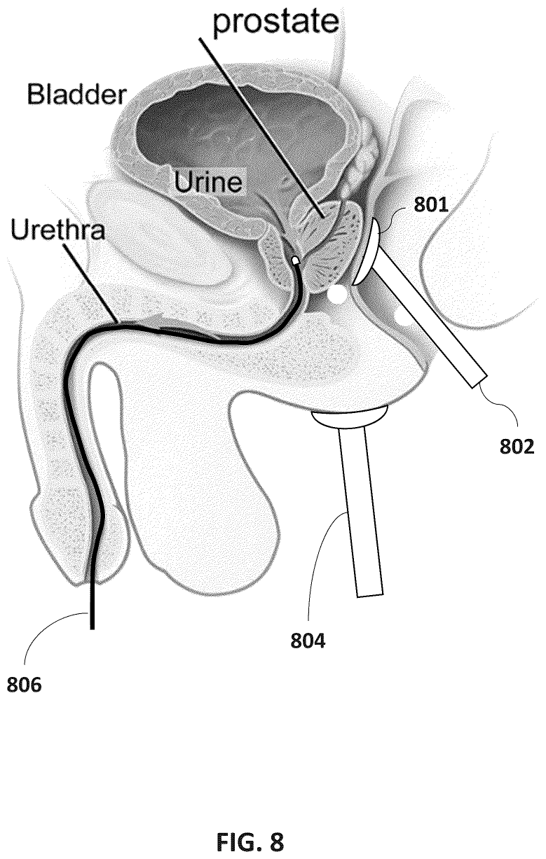

In some embodiments, the method comprises insonating the whole prostate and/or substantially the whole prostate in a one-stage procedure. Alternatively or additionally, the method comprises insonating the prostate in more than one step, wherein each step comprises insonating a portion and/or a region of the prostate and collecting a sample therefrom. The stepped procedure potentially allows to detect a location of dysplastic cells or pancreatic cancer cells within the prostate. In some embodiments, the stepped insonation of the prostate is achieved by adjusting the positioning of the ultrasound probe and/or adjusting the ultrasound focus to insonate a different region of the prostate at each stage. In some embodiments, an ultrasound probe (for example, probe 802, 804 and/or 806, described in FIG. 8), comprises a plurality of separate transducers, each can be aimed and/or focused on a different portion of the prostate. In some embodiments, the probe comprises a high-intensity focused ultrasound.

Reference is now made to FIG. 7, showing a schematic cross-sectional view of a prostate follicle 440 and a segment of a prostate duct (which drains eventually into the urethra), according to an example embodiment of the invention. As shown, a layer of epithelial cells 442 (including basal, luminal and neuroendocrine) are situated atop a basement membrane 444. In embodiments of the invention, a method is provided for dislodging epithelial cells (for example cells 448), epithelial cells group(s) (for example cells groups 450) and/or epithelial tissue fragment(s) which then expelled in ejaculate and/or drain into the bladder and are subsequently expelled in urine. In embodiments of the invention, the method includes the steps of insonating a prostate of the subject with an ultrasound probe with an amount of ultrasonic energy at a predetermined point in the prostate to elicit exfoliation of cells into the segment of a prostate duct 446 and eventually into urethra of the subject.

Reference is now made to FIG. 8, showing a schematic cross-sectional view of a patient's pelvic region and simplified drawings of ultrasound probes interacting with the patient's prostate, according to an example embodiment of the invention.

In some embodiments, ultrasound devices constructed and/or programmed to employ methods for producing cytopathologic cell samples from a prostate, (as described in this document, for example in FIG. 6), comprising an ultrasound probe configured for insonating the prostate. In some embodiments, said probe is a variant of probe 410, adjusted for the prostate, for example, sized and/or shaped to fit in the pelvic region.

The ultrasound probe comprises a transducer, a power source, and a control which operates the transducer using a power supply from the power source. In some embodiments, the transducer is configured to send focused controllable beams.

In some embodiments, said probe comprises a transrectal ultrasound probe 802. The probe is configured to be inserted into the patient's rectum and reach the prostate. In some embodiments, transrectal ultrasound probe 802 comprises a concave contact surface 801 that fits the shape of the prostate surface. In some embodiments, transrectal ultrasound probe 802 potentially allows to insonate a whole prostate and/or substantially a whole prostate. Alternatively or additionally, transrectal ultrasound probe 802 is configured to insonate portions/regions of the prostate, for example, in some embodiments, a contact surface of the probe is divided into areas, each area for insonating a different portion of the prostate. In some embodiments, the prob comprises a plurality of separate transducers, potentially allowing to test only a part of the prostate at the time for detecting abnormalities at said part. Testing the prostate by testing only a part thereof at a time potentially allows to detect a location of an abnormality within the prostate.

In some embodiments, the ultrasound device comprises a transperineal ultrasound probe 804. Transperineal ultrasound probe 804 is configured to be positioned on the perineum and to be aimed for applying ultrasound waves toward the prostate.

Applying the ultrasound waves externally (e.g., probe 804 contacting the perineum) has the potential advantage of reducing the patient's discomfort and/or pain.

In some embodiments, the ultrasound device comprises an ultrasound catheter probe 806, configured to be inserted into the patient's urethra. Probe 806 comprises an ultrasound transducer at a tip thereof, connected to a flexible elongated shaft, that allows it to be inserted into the urethra and navigated through the urinary tract to reach the prostate. In some embodiments, the transducer at the tip of probe 806, is used for insonating the prostate in more than one stage. In each stage, another portion (e.g. region of the prostate is insonated and at least one sample is collected therefrom. The plurality of stages allows to identify a location of abnormal cells, if detected. In some embodiments, probe 806 comprises more than one transducer at the tip and/or along the shaft thereof, allowing to insonate wider portions of the prostate and/or the whole and/or substantially the whole prostate (e.g., in a single stage).

In some embodiments, the probe (e.g., 802, 804 and/or 806) comprises an imaging system for monitoring the position and/or direction of the probe. In some embodiments, the imaging system is located at the tip of the prob (for example tip 801 of prob 802, at the tip of probe 806 and/or at the contact surface of probe 804), and/or located near the transducer of the probe. In some embodiments, the imaging system comprises an ultrasound imaging system. In some embodiments, the transducer of the probe is used for imaging in addition to insonating, optionally the imaging is performed between pulses of insonation. The monitoring potentially ensures that the probe is aimed toward the prostate and/or focused within the prostate. In some embodiments, the imaging system is connected to a control that alerts and/or stops applying ultrasound waves if deviation from the prostate is detected. For example, if the patient and/or the physician move, or due to internal organ displacement, for example as a result of the bladder being filled. Monitoring the direction of the probe has the potential advantage of reducing and/or avoiding the risk of impairing nerves, blood vessels and/or organs (such as the bladder) surrounding the prostate as a result of misdirection of probe. In some embodiments, the imaging system comprises a controller for identifying the prostate, optionally, the prostate is initially marked by a human operator and/or an AI (artificial intelligence) component.

In some embodiments, the ultrasound probe (e.g. probe 802, 804, and/or 806) comprises an electroejaculation device/component. In some embodiments, the probe comprises electrode(s) that deliver electrical stimulation and a control for operating and adjusting the intensity and frequency of the electrical pulses.

In some embodiments, the ultrasound probe (e.g. probe 802, 804, and/or 806) comprises a prostate stimulator (e.g., a prostate massager), configured to apply gentle pressure and/or vibrations to the prostate gland, for promoting prostate secretion. For example, probe 802 comprises said stimulator at a tip thereof.

It is expected that during the life of a patent maturing from this application many relevant ultrasound transducers will be developed and the scope of the term ultrasound is intended to include ultrasound produced by all such new technologies a priori.

It is expected that during the life of a patent maturing from this application many relevant ultrasound contrast materials will be developed and the scope of the term ultrasound contrast material is intended to include all such new ultrasound contrast materials a priori.

As used herein with reference to quantity or value, the terms approximately and “about” mean “within ±25% of”.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Various embodiments and aspects of the present disclosure as delineated hereinabove and as claimed in the claims section below find experimental support in the following examples.

### Examples

Reference is now made to the following examples, which together with the above descriptions illustrate some embodiments of the disclosure in a non-limiting fashion.

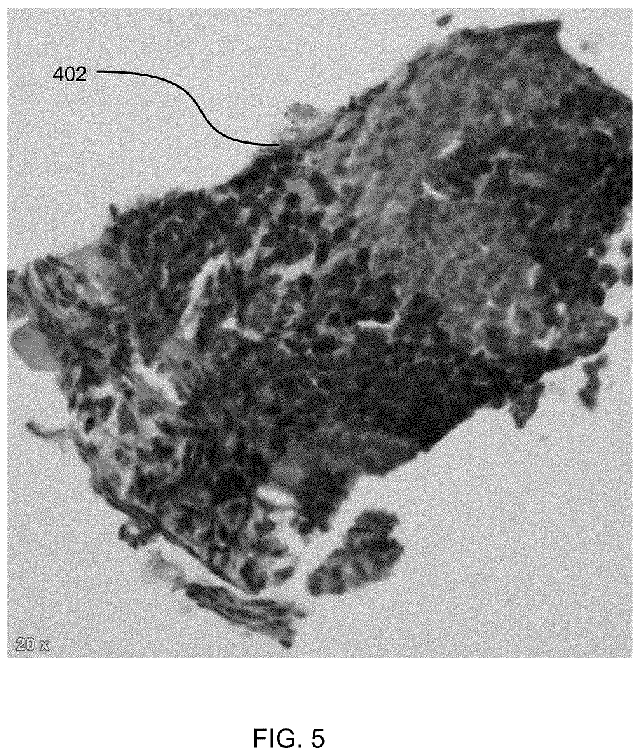

Reference is now made to FIG. 5, which is an image of a group of contiguous cells obtained by an example embodiment of the invention.

FIG. 5 shows a group 402 or patch 402 of pancreatic duct cells.

### EXPERIMENTAL RESULTS

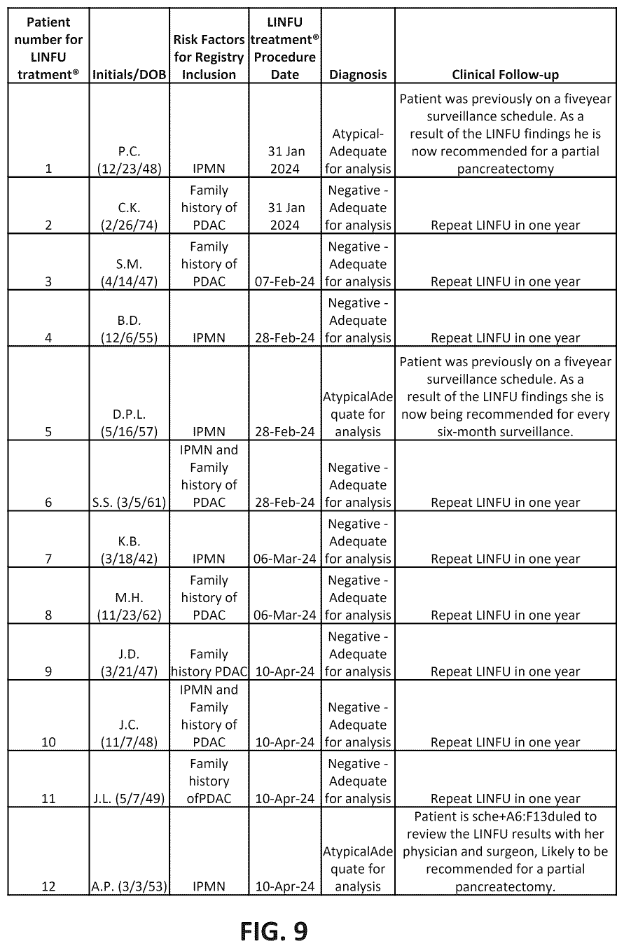

Reference is now made to FIG. 9, which is a table of experimental results of a clinical registry (at Sarasota Memorial Hospital), by an example embodiment of the invention.

12 patients were subjected to ultrasound insonation in a protocol that included insonating the pancreas with higher intensities of partially focused ultrasound waves without microbubble contrast agent during a period of time to generate shear waves followed by insonation using lower intensities of less focused ultrasound waves combined with microbubbles in accordance with embodiments of the invention set forth herein. Pancreatic cell samples collected from all 12 patients contained sufficient cells to render the results “adequate for analysis.” Atypical cells were detected in three patients (patients number 1, 5 and 12)—which changed the course of treatment for these patients. Patient number 1 was previously on a five-year surveillance schedule. As a result of the procedure findings, he is recommended for a partial pancreatectomy. Patient number 5 was previously on a five-year surveillance schedule. As a result of the procedure findings, she is being recommended for every six-month surveillance. Patient number 12 is scheduled to review the procedure results with her physician and surgeon, Likely to be recommended for a partial pancreatectomy. The other patients are instructed to repeat the procedure in one year.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of inducing exfoliation of cells and molecules from a targeted epithelium, the method comprising:
insonating a target tissue with a high energy mechanical index (MI) at a level greater than 0.4 focused beam to generate shear waves within the tissue.
